(19) United States
(12) Patent Application Publication
(10) Pub. No.: US 2010/0298685 A1
(43) Pub. Date: Nov. 25, 2010

(54) ADAPTIVE INSULIN DELIVERY SYSTEM

(75) Inventors: Gary A. Hayter, Oakland, CA (US); Erwin S. Budiman, Fremont, CA (US); Charles Wei, Fremont, CA (US)

Correspondence Address: Fulwider Patton LLP (ADC), 6060 Center Drive, 10th Floor, Los Angeles, CA, 90045, US

(73) Assignee: ABBOTT DIABETES CARE INC., ALAMEDA, CA (US)

(21) Appl. No.: 12/785,196

(22) Filed: May 21, 2010

Related U.S. Application Data
(60) Provisional application No. 61/180,767, filed on May 22, 2009.
(60) Provisional application No. 61/180,627, filed on May 22, 2009.
(60) Provisional application No. 61/180,649, filed on May 22, 2009.
(60) Provisional application No. 61/180,774, filed on May 22, 2009.
(60) Provisional application No. 61/180,700, filed on May 22, 2009.

(51) Int. Cl. A61B 5/145; A61M 5/00

(52) U.S. Cl. 600/365; 604/500

(57) ABSTRACT

A proactive system and method in which levels of glucose are monitored after a meal signal and compared to a safe range. If a monitored glucose level is outside the safe range, a post-prandial vertex of the glucose level is identified and an action is provided to more rapidly return the glucose level to a target level within the safe range than if no action was provided. In another aspect a control parameter in an IDM system is adjusted by determining a performance metric of the system as a function of the levels of glucose and a medication administration signal over a first window of time; and, if the performance metric is outside an expected range, adjusting the control parameter to adjust an amount of medication and to bring the performance metric inside the expected range.

### CROSS-REFERENCES TO RELATED APPLICATIONS

This application claims the benefit of U.S. application Ser. No. 61/180,767, filed May 22, 2009 which is incorporated herein by reference in its entirety.

This application is also related to U.S. application Ser. No. ______ entitled “Safety Features For Integrated Insulin Delivery System,” (U.S. Provisional Application No. 61/180,627, filed May 22, 2009); U.S. application Ser. No. ______ entitled “Usability Features For Integrated Insulin Delivery System,” (U.S. Provisional Application No. 61/180,649, filed May 22, 2009); U.S. application Ser. No. ______ entitled “Safety Layer for Integrated Insulin Delivery System,” (U.S. Provisional Application No. 61/180,774); and U.S. application Ser. No. ______ entitled “Methods for Reducing False Hypoglycemia Alarm Occurrence,” (U.S. Provisional Application No. 61/180,700, filed May 22, 2009).

### BACKGROUND

The invention is generally related to systems and methods for control over the delivery of medication and more particularly, to an adaptive system for controlling the delivery of medication.

Diabetes is a metabolic disorder that afflicts tens of millions of people throughout the world. Diabetes results from the inability of the body to properly utilize and metabolize carbohydrates, particularly glucose. Normally, the finely-tuned balance between glucose in the blood and glucose in bodily tissue cells is maintained by insulin, a hormone produced by the pancreas which controls, among other things, the transfer of glucose from blood into body tissue cells. Upsetting this balance causes many complications and pathologies including heart disease, coronary and peripheral artery sclerosis, peripheral neuropathies, retinal damage, cataracts, hypertension, coma, and death from hypoglycemic shock.

In patients with insulin-dependent diabetes, the symptoms of the disease can be controlled by administering additional insulin (or other agents that have similar effects) by injection or by external or implantable insulin pumps. It is understood that throughout this document, the terms “patient” and “user” are used interchangeably. The “correct” insulin dosage is a function of the level of glucose in the blood. Ideally, insulin administration should be continuously readjusted in response to changes in glucose level.

Presently, systems are available for monitoring glucose levels by implanting a glucose sensitive probe into the user. Such probes measure various properties of blood or other tissues, including optical absorption, electrochemical potential and enzymatic products. The output of such sensors can be communicated to a hand held device that is used to calculate an appropriate dosage of insulin to be delivered into the blood stream in view of several factors, such as a user's present glucose level, insulin usage rate, carbohydrates consumed or to be consumed and exercise, among others. These calculations can then be used to control a pump that delivers the insulin, either at a controlled “basal” rate, or as a “bolus.” When provided as an integrated system, a continuous glucose monitor (“CGM”), a pump, and a control means work together to provide continuous glucose monitoring and insulin pump control. Integrated diabetes management (“IDM”) systems, whether implemented as a fully closed-loop, semi closed-loop, or an open loop system, are designed to insure the accuracy of the glucose monitor and to protect the user from either under- or over-dosage of insulin, as well as to provide improved usability, control, and safety

Certain activities performed by a person suffering from diabetes can cause a significant change in the level of glucose of that person. Typically, diabetic users have a “safe range” within which their glucose should be confined. It is preferable for the user to maintain his or her glucose within this range at all times, but if the glucose level does go outside this range, it is also preferable for the user to take immediate steps to cause his or her glucose level to return to the safe range as quickly as possible. This is true for both hyperglycemia and hypoglycemia.

One of the activities that causes glucose to vary significantly is the consumption of a meal. This activity can add a large amount of glucose to the user's bloodstream in a short period of time which can result in the user's glucose rising to a level outside the safe range. In an attempt to avoid such an occurrence, many users give themselves an injection of insulin prior to the meal for the purpose of controlling a post prandial rise in glucose. If the contents of the meal were accurately identified (for instance, the level of carbohydrates, fat, and other components), an insulin calculator may accurately advise the user on the correct pre-prandial insulin injection. However this is not always the case. The user may not have been clear on the contents of the meal, and the amount of pre-prandial insulin may be much less or much more than needed. It would thus be desirable to specifically monitor the user's glucose level after the meal so that immediate steps can be taken if the glucose level is out of the safe range.

When a user's glucose is outside the safe range, negative effects on the user's physiology can occur due to the excess or lack of glucose. It would also be desirable to provide a user with steps to take to return to the safe range as soon as possible. Such steps would need to take into account insulin-on-board and other user specific information.

A typical strategy for reducing glycemic exposure is to use a high glucose alarm, that is, notifying a user when his or her glucose exceeds a certain range of values determined to be safe. The user will typically self-treat to reduce the glucose when the high glucose alarms sounds. However, this often means that preemptive pump control processes cannot start until the high glucose alarm is triggered. Also, glucose alarms tend to be fairly high (for example, 240 mg/dL). If the glucose hovers above the target glucose, but below the high glucose threshold, then, after a meal, the user may not take action unless he or she sees a high glucose value, which can be too late to keep the glucose value below the high glucose threshold. Another common weakness of passive systems is to just display the CGM value and wait for the user to act. For instance, after a meal and after performing a food and correction bolus, a user-user will typically initiate self-analysis to see if the bolus is amount is appropriate. The user can then look again at the value 90 minutes after the meal assuming that this is the peak, but the peak may not occur until later. Without knowing the peak, it is difficult to know if the user's intervention is adequate or not.

Hence, those skilled in the art have recognized a need for a more proactive system and method to identify that a user's glucose level is outside the user's safe range, and provide steps to rapidly return the user's glucose level to the safe range. A CGM system may increase the probability that the user will see the value and act, but a need exists for a system that facilitates the process by issuing an earlier proactive alert as soon as the peak is detected and immediately provides the user with information regarding a dosing of medication to quickly return the user's glucose levels to a normal state within the user's safe range.

Another very important part in diabetes management is accurately setting the system parameters that are considered by a IDM system in managing the glucose levels of a user. Some of these parameters are associated with the physiology of the person with diabetes. Closed loop systems for glucose control may also include models and/or control parameters that are fixed while the closed loop system is operating. These fixed parameters may be chosen for an “average” population—and the glucose control performance (for example, accuracy, safety margin, lower false alarms, etc) may be improved if these fixed parameters are tailored specifically for the user. However, these fixed parameters may change over time. For example, one of which is insulin sensitivity—the response that a particular user has to insulin—otherwise known as insulin resistance which not only vary from person to person but depend on day-to-day circumstances of the individual. Other variable factors may include a user's glucose safe range. While some systems may provide for limited manual modification of some parameters, most parameters are typically periodically adjusted under the guidance of a user's HCP. Infrequent contact with HCPs and/or lack of attention to parameters ultimately leads to use of control parameters in IDM systems that are not ideal and/or our of tolerance. Therefore, those skilled in the art have recognized a need for a system and method for dynamically revising such system parameters in real time based on historical and other user data, so that glucose may be more effectively managed by users. The present invention fulfills these needs and more.

### SUMMARY OF THE INVENTION

The present invention is directed to a system for integrated diabetes management. In some aspects, the invention includes a system for proactively monitoring glucose levels, including a sensor that measures an indication of glucose and provides levels of glucose over a monitoring time period, a memory having stored therein a safe range of glucose, a device that provides a meal signal indicating that a meal has been consumed, the device also providing a medication administration signal indicating an amount of medication and a time that it was administered, and a processor configured to receive the meal signal, levels of glucose, and the safe range, wherein upon receiving the meal signal. In this aspect, the processor is further configured to monitor the levels of glucose beginning after the meal signal, compare the levels of glucose to the safe range, and, if a monitored glucose level is outside the safe range, determine a post-prandial vertex of the glucose level, and, once the vertex is determined, provide an action to return the glucose level to a target glucose level within the safe range, wherein the action includes consideration of a user parameter.

In one aspect, the system the vertex is a peak and the action provided by the processor includes calculating a dose of medication sufficient to reduce the glucose level more rapidly to the target glucose level within the safe range and to decrease an amount of high glucose exposure than if the action was not taken. In another aspect, the vertex is a valley and the action provided by the processor includes calculating an amount of carbohydrates sufficient to increase the glucose level more rapidly to the target glucose level within the safe range than if the action was not taken. In a further aspect, the vertex is a valley and the action by the processor includes calculating a reduction in a basal rate of a medication administration sufficient to raise the glucose level more rapidly to the target glucose level within the safe range than if the action was not taken.

In some aspects, the processor is configured to delay monitoring the levels of glucose for a selected time period after receiving the meal signal. In some aspects, the the processor is configured to determine the vertex by comparing a first and a second glucose level trend, the vertex being at the point at which the first and second glucose level trends diverge. In a further aspect, the processor is configured to determine the vertex by comparing a first and a second glucose rate of change, the vertex being at the point at which the signs of the first and second glucose rate of change diverge.

In some aspects, the processor is further configured to identify a first and second set of sensed glucose readings over at least a portion of the monitoring time period to determine a first and second glucose level trend, the vertex being determined by comparing a first and a second glucose level trend, and to calculate a first and second set of smoothed glucose values representative of the first and second set of sensed glucose readings prior to determining the first glucose level trend, and to determine the first and second glucose level trend as a function of first and second set of smoothed glucose values. In one aspect, the first and second glucose level trend is a trend in a first and a second glucose rate of change. In some aspects, the user parameter is selected from a group consisting of an insulin action time, a level of insulin-on-board, an insulin sensitivity factor

In other aspects, the present invention includes a method for proactively monitoring glucose levels, the method including measuring an indication of glucose and providing levels of glucose over a monitoring time period, storing in a memory a safe range of glucose, providing a meal signal indicating that a meal has been consumed, providing a medication administration signal indicating an amount of medication and a time that it was administered, receiving the meal signal, levels of glucose, and the safe range, wherein upon receiving the meal signal, monitoring the levels of glucose beginning after the meal signal, comparing the levels of glucose to the safe range, and, if a monitored glucose level is outside the safe range, determining a post-prandial vertex of the glucose level, and once the vertex is determined, providing an action to return the glucose level to a target glucose level within the safe range, wherein the action includes consideration of a user parameter.

In some aspects of the method, the vertex is a peak and providing the action includes calculating a dose of medication sufficient to reduce the glucose level more rapidly to the target glucose level within the safe range and to decrease an amount of high glucose exposure than if the action was not taken. In some aspects, the vertex is a valley and providing the action includes calculating an amount of carbohydrates sufficient to increase the glucose level more rapidly to the target glucose level within the safe range than if the action was not taken. In further aspects, the vertex is a valley and providing the action includes calculating a reduction in a basal rate of a medication administration sufficient to raise the glucose level more rapidly to the target glucose level within the safe range than if the action was not taken.

In some aspects, the method for proactively monitoring glucose levels further includes delaying monitoring the levels of glucose for a selected time period after receiving the meal signal. In at least one aspect, the vertex is determined by comparing a first and a second glucose level trend, the vertex being at the point at which the first and second glucose level trends diverge. In some aspects, the vertex is determined by comparing a first and a second glucose rate of change, the vertex being at the point at which the signs of the first and second glucose rate of change diverge.

In some aspects, the method for proactively monitoring glucose levels further includes identifying a first and second set of sensed glucose readings over at least a portion of the monitoring time period to determine a first and second glucose level trend, the vertex being determined by comparing a first and a second glucose level trend, and calculating a first and second set of smoothed glucose values representative of the first and second set of sensed glucose readings prior to determining the first glucose level trend, and to determine the first and second glucose level trend as a function of first and second set of smoothed glucose values. In one aspect, the first and second glucose level trend is a trend in a first and a second glucose rate of change. In one aspect, the user parameter is selected from a group consisting of an insulin action time, a level of insulin-on-board, and an insulin sensitivity factor.

In some aspects, the present invention includes a method for integrated diabetes management, including receiving at a controller a meal indication input, receiving at the controller and after the meal indication input a first set of sensed glucose readings in a user from a continuous glucose sensor, receiving at the controller a second set of sensed glucose readings in the user from the continuous glucose sensor, wherein the second set of sensed glucose readings begin later in time than the first set of sensed glucose readings and at least one of the second set of sensed readings is above a high glucose threshold, recording the first and second set of glucose readings on a memory medium, determining a first glucose level trend in the user as a function of the first set of glucose signals, determining a second glucose level trend in the user as a function of the second set of glucose signals, sending a signal indicative of a user glucose level peak from the controller to a display when the first glucose level trend and second glucose level trend diverge; and wherein the signal includes an amount of insulin required to reduce an insulin action time necessary to reduce a third set of sensed glucose readings below the high glucose threshold and to minimize a high glucose exposure. In some aspects, the first glucose level trend is a first rate of change in the first set of sensed glucose readings in the user, and the second glucose level trend is a second rate of change in the second set of sensed glucose readings in the user.

In some aspects, the method further includes calculating a first set of smoothed glucose values representative of the first set of sensed glucose readings prior to determining the first glucose level trend, wherein the first glucose level trend is determined as a function of first set of smoothed glucose values, and calculating a second set of smoothed glucose value representative of the second set of sensed glucose readings prior to determining the second glucose level trend; wherein the second glucose level trend is determined as a function of second set of smoothed glucose values. In some aspects, the first glucose level trend is a value in the second set of smoothed glucose values.

In some aspects, the present invention includes a method for adjusting a control parameter used in an integrated diabetes management (IDM) system, the method including storing in a memory a control parameter, providing a medication administration signal representative of an amount of medication as a function of the control parameter, measuring levels of glucose over a monitoring time period, determining a performance metric as a function of the levels of glucose and the medication administration signal over a first window of time; and, if the performance metric is outside an expected range, adjusting the control parameter to adjust the amount of medication and to bring the performance metric inside the expected range. In some aspects, the expected range is determined as a function of an empirical series of performance metrics over a selected window of time. In some aspects, the selected window starts before a beginning of the first window. In some aspects, the first window at least partially overlaps the selected window in time. In other aspects, the first and selected window do not overlap in time.

In some aspects of the method, the performance metric includes an average glucose expected from a present time to a future time. In some aspects, the performance metric includes a slope of the levels of glucose over a selected window of time. Other examples of performance metrics include, but are not limited to, a level of glucose at a point in the future, a number and/or time a particular value is out of tolerance, a number and/or time of hypoglycemic or hyperglycemic events, and a number and/or amount of bolus corrections.

In some aspects, the method for adjusting a control parameter further includes providing a control limit on the control parameter, monitoring a set of control parameters, determining one or more physiological events over a selected window of time, and, if the one or more physiological events is outside an unacceptable level of physiological events, adjusting the control limit to avoid a further unacceptable physiological event. In some aspects, the control limit is determined as a function of a scatter of the set of control parameters over a selected time period. In some aspects, the unacceptable physiological events includes a number of hypoglycemic events in a user. In other aspects, the unacceptable physiological events includes a number of hyperglycemic events in a user.

In some aspects, the method for adjusting a control parameter further includes, if, over a selected window of time, consecutively computed insulin commands are negative for more than a predetermined duration, providing an alarm including an indication of a high likelihood of a hypoglycemic event. In some aspects, the method further includes providing a request to enter a manual glucose reading using a strip port, wherein the control parameter is adjusted as a function of the manual glucose reading. In further aspects, the method includes, if the manual glucose reading confirms the consecutively computed insulin command, providing a second alarm including a suggestion to ingest an amount of carbohydrates.

In some aspects, the method includes, if, over a selected window of time, a first contiguous area formed by consecutively computed insulin commands exceeds a second area formed by an integral of a predetermined duration, providing an alarm including an indication of a high likelihood of a hypoglycemic event. In some aspects, the method further includes providing a request to enter a manual glucose reading using a strip port, wherein the control parameter is adjusted as a function of the manual glucose reading. In further aspects, the method includes, if the manual glucose reading confirms the consecutively computed insulin command, providing a second alarm including a suggestion to ingest an amount of carbohydrates.

In some aspects, the present invention includes a system for adjusting a control parameter used in an integrated diabetes management (IDM) system, the system including a memory having a control parameter stored thereon, a device configured to provide a medication administration signal representative of an amount of medication as a function of the control parameter, the device being further configured to measure levels of glucose over a monitoring time period, a processor configured to determine a performance metric as a function of the levels of glucose and the medication administration signal over a first window of time, such that, if the performance metric is outside an expected range, the processor is further configured to adjust the control parameter to adjust the medication administration signal and to bring the performance metric inside the expected range. In some aspects, the processor is further configured to determine the expected range as a function of an empirical series of performance metrics over second window of time. In some aspects, the selected window starts before a beginning of the first window. In some aspects, the first window at least partially overlaps the selected window in time. In other aspects, the first and selected window do not overlap in time.

In some aspects of the system, the performance metric includes an average glucose expected from a present time to a future time. In some aspects, the performance metric includes a slope of the levels of glucose over a selected window of time. Other examples of performance metrics include, but are not limited to, a level of glucose at a point in the future, a number and/or time a particular value is out of tolerance, a number and/or time of hypoglycemic or hyperglycemic events, and a number and/or amount of bolus corrections.

In some aspects, the system for adjusting a control parameter further includes providing a control limit on the control parameter, monitoring a set of control parameters, determining one or more physiological events over a selected window of time, and, if the one or more physiological events is outside an unacceptable level of physiological events, adjusting the control limit to avoid a further unacceptable physiological event. In some aspects, the control limit is determined as a function of a scatter of the set of control parameters over a selected time period. In some aspects, the unacceptable physiological events includes a number of hypoglycemic events in a user. In other aspects, the unacceptable physiological events includes a number of hyperglycemic events in a user.

In some aspects, the system for adjusting a control parameter further includes, if, over a selected window of time, consecutively computed insulin commands are negative for more than a predetermined duration, providing an alarm including an indication of a high likelihood of a hypoglycemic event. In some aspects, the system further includes providing a request to enter a manual glucose reading using a strip port, wherein the control parameter is adjusted as a function of the manual glucose reading. In further aspects, the system includes, if the manual glucose reading confirms the consecutively computed insulin command, providing a second alarm including a suggestion to ingest an amount of carbohydrates.

In some aspects, the system for adjusting a control parameter further includes, if, over a selected window of time, a first contiguous area formed by consecutively computed insulin commands exceeds a second area formed by an integral of a predetermined duration, providing an alarm including an indication of a high likelihood of a hypoglycemic event. In some aspects, the system includes providing a request to enter a manual glucose reading using a strip port, wherein the control parameter is adjusted as a function of the manual glucose reading. In further aspects, the system includes, if the manual glucose reading confirms the consecutively computed insulin command, providing a second alarm including a suggestion to ingest an amount of carbohydrates.

In some aspects, the present invention includes a method for integrated diabetes management, including providing a controller with a control parameter, sampling at the controller a first set of insulin delivery commands over a first window of time, sampling at the controller a second set of insulin delivery commands over a second window of time, wherein the first and second insulin delivery commands provide information to deliver an amount of insulin and are determined as a factor of a difference between a sensed glucose value and a target glucose value and a control parameter, determining a first performance metric as a function of the first set of insulin delivery commands, determining a second performance metric as a function of the second set of insulin delivery commands, adjusting the control parameter as a function of the first and second performance metric to generate an adjusted control parameter, determining a future insulin delivery command as a function of the adjusted control parameter; and, graphically displaying information representative of the future insulin delivery command on a graphic display. In some aspects, the first and second insulin delivery commands are further determined as a factor of a difference between the latest CGM rate of change and a target rate of change.

In further aspects, the method for integrated diabetes management includes delivering a first insulin amount to a user, and delivering a second insulin amount to the user based on the second delivery command, wherein the second insulin amount is based on the difference between a present value of glucose and a target value of glucose. In some aspects, the present value is a present CGM value and the target value is a target CGM value. In other aspects, the present value is a present CGM rate of change, and the target value is a target CGM rate of change. In further aspects, the present value is a present insulin-on-board, and the target value is a target insulin-on-board.

In some aspects, the method includes tuning the target value to correlate to a time period or physical condition. In yet further aspects, the method includes determining an insulin delivery command as a function of the performance metric and the adjusted control parameter, prompting a user at a terminal to confirm the insulin delivery amount, and delivering the insulin delivery amount to a user upon receiving a confirmation from the user at the terminal.

The features and advantages of the invention will be more readily understood from the following detailed description which should be read in conjunction with the accompanying drawings.

### BRIEF DESCRIPTION OF THE DRAWINGS

FIG. 1 is a schematic diagram illustrating an exemplary embodiment of an electronic device and its various components in operable communication with one or more medical devices, such as a glucose monitor or drug delivery pump, and optionally, in operable communication with a remote computing device.

FIG. 2 depicts an integrated diabetes management (“IDM”) system in accordance with aspects of the present invention;

FIG. 3A is a graph of glucose level versus time showing a safe range for glucose in dashed horizontal lines, a target level of glucose within that safe range also in a dashed horizontal line, and further presenting a solid-line curve of actual glucose measurements of a user showing that the glucose exceeded the upper limit of the safe range, reached a peak or “vertex,” and in a solid line showing a more rapid return to the target glucose level than the dashed line, and also showing the area under the curve in diagonal lines indicating the time and level that the patent was outside the safe range; and

FIG. 3B is a graph similar to FIG. 3A showing a portion of the safe range, the upper limit of the safe range, and the curve of actual user glucose level over time, but also showing two trend arrows before and after the vertex, one of which indicates a positive slope and the other of which indicates a negative slope, the point where the slope changes from positive to negative being the peak (vertex) of the user's glucose curve.

### DETAILED DESCRIPTION OF THE PREFERRED EMBODIMENTS

For the purposes of promoting an understanding of the principles of the invention, reference will now be made to a number of illustrative embodiments shown in the attached drawings and specific language will be used to describe the same.

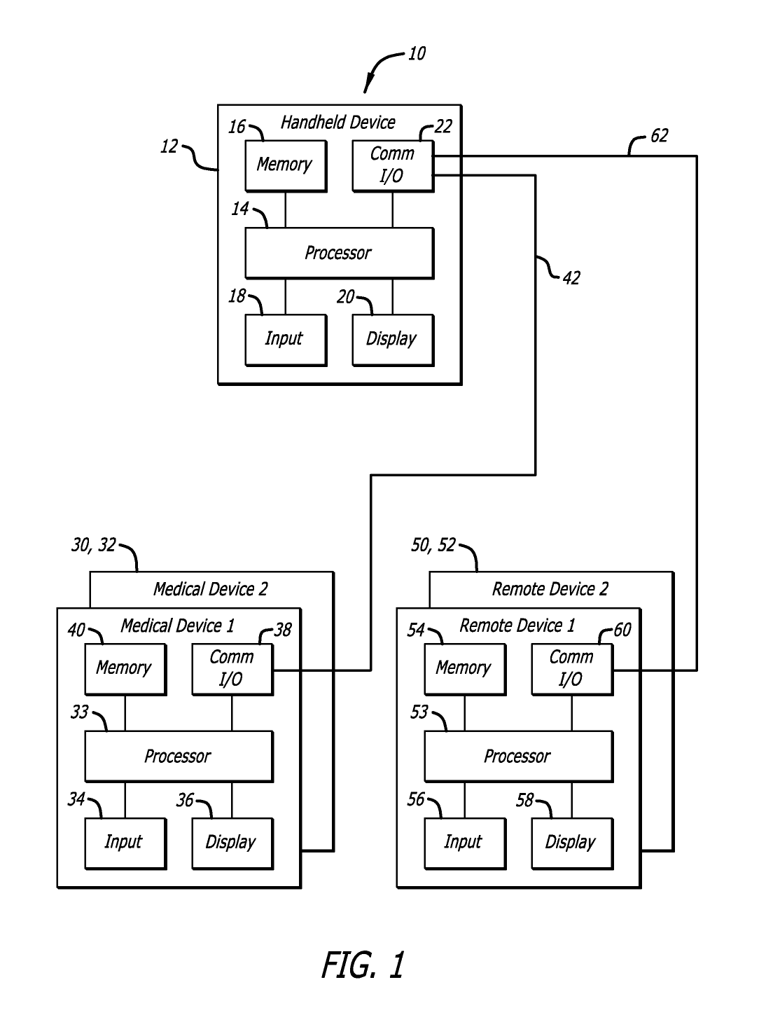

Referring now to FIG. 1, a block diagram of one illustrative embodiment of a system 10 for monitoring, determining and/or providing drug administration information is shown. It should be understood for the purpose of the depicted embodiments that system 10 is depicted as an IDM system including a CGM sensor device, an insulin pump, and a control means (for example, a handheld device) that may work together to provide continuous glucose monitoring and insulin pump control, and that may further be implemented as a fully closed-loop, semi closed-loop, or an open loop system. In the illustrated embodiment, the system 10 includes an electronic device 12 having a processor 14 in data communication with a memory unit 16, an input device 18, a display 20, and a communication input/output unit 24. The electronic device 12, which may be handheld, may be provided in the form of a general purpose computer, central server, personal computer (PC), laptop or notebook computer, personal data assistant (PDA) or other hand-held device, external infusion pump, glucose meter, analyte sensing system, or the like. The electronic device 12 may be configured to operate in accordance with one or more operating systems including for example, but not limited to, WINDOWS, Unix, LINUX, BSD, SOLARIS, MAC OS, or, an embedded OS such as ANDROID, PALM OS, WEBOS, eCOS, QNX, or WINCE, and may be configured to process data according to one or more internet protocols for example, but not limited to, NetBios, TCP/IP and APPLETALK. The processor 14 is microprocessor-based, although the processor 14 may be formed of one or more general purpose and/or application specific circuits and operable as described hereinafter. The memory unit 16 includes sufficient capacity to store operational data, one or more software algorithms executable by the processor 14, and other user inputted data. The memory unit 16 may include one or more memory or other data storage devices.

Display 20 is also included for viewing information relating to operation of the device 12 and/or system 10. Such a display may be a display device including for example, but not limited to, a light emitting diode (LED) display, a liquid crystal display (LCD), a cathode ray tube (CRT) display, or the like. Additionally, display 20 may include an audible display configured to communicate information to a user, another person, or another electronic system having audio recognition capabilities via one or more coded patterns, vibrations, synthesized voice responses, or the like. Additionally, display 20 may include one or more tactile indicators configured to display tactile information that may be discerned by the user or another person.

Input device 18 may be used in a manner to input and/or modify data. Input device 18 may include a keyboard or keypad for entering alphanumeric data into the processor 14. Such a keyboard or keypad may include one or more keys or buttons configured with one or more tactile indicators to allow users with poor eyesight to find and select an appropriate one or more of the keys, and/or to allow users to find and select an appropriate one or more of the keys in poor lighting conditions. Additionally, input device 18 may include a mouse or other point and click device for selecting information presented on the display 20. Additionally, input device 18 may include display 20, configured as a touch screen graphical user interface. In this embodiment, the display 20 includes one or more selectable inputs that a user may select by touching an appropriate portion of the display 20 using an appropriate implement.

Input device 18 may also include a number of switches or buttons that may be activated by a user to select corresponding operational features of the device 12 and/or system 10. Input device 18 may also be or include voice-activated circuitry responsive to voice commands to provide corresponding input data to the processor 14. The input device 18 and/or display 20 may be included with or separate from the electronic device 12.

System 10 may also include a number of medical devices 30, 32 which carry out various functions, for example, but not limited to, monitoring, sensing, diagnostic, communication and treatment functions. In such embodiments, any of the one or more of the medical devices 30, 32 may be implanted within the user's body, coupled externally to the user's body (for example, such as an infusion pump), or separate from the user's body. In some embodiments, medical devices 30, 32 are controlled remotely by electronic device 12. Additionally, one or more of the medical devices may be mounted to and/or form part of the electronic device 12. For example, in some embodiments, electronic device 12 includes an integrated glucose meter or strip port and is configured to receive a signal representative of a glucose value and display the value to a user. Electronic device 12 may further be configured to be used to calibrate a continuous glucose monitor (CGM) or for calculating insulin amounts for bolus delivery. Typically, the medical devices 30, 32 are each configured to communicate wirelessly with the communication I/O unit 22 of the electronic device 12 via one of a corresponding number of wireless communication links. Wireless communication is preferable when medical device 30, 32 is configured to be located on a remote part of the body, for example, in an embodiment wherein medical device 30, 32 is a continuous glucose monitor (CGM) or sensor, or insulin pump, worn under clothing.

Electronic device 12 communicates with medical device 30, 32 via a wireless protocol, or, in some embodiments, is directly connected via a wire. The wireless communications between the various components of the system 10 may be one-way or two-way. The form of wireless communication used may include, but should not be limited to, radio frequency (RF) communication, infrared (IR) communication, Wi-Fi, RFID (inductive coupling) communication, acoustic communication, capacitive signaling (through a conductive body), galvanic signaling (through a conductive body), BLUETOOTH, or the like. Electronic device 12 and each of the medical devices 30, 32 include circuitry for conducting such wireless communications circuit. In another embodiment, one or more of the medical devices 30, 32 may be configured to communicate with electronic device 12 via one or more serial or parallel configured hardwire connections therebetween.

Each of the one or more medical devices 30, 32 may include one or more of a processing unit 33, input 34 or output 36 circuitry and/or devices, communication ports 38, and/or one or more suitable data and/or program storage devices 40. It may be understood that not all medical devices 30, 32 will have the same componentry, but rather will only have the components necessary to carry out the designed function of the medical device. For example, in one embodiment, a medical device 30, 32 may be capable of integration with electronic device 12 and thus omit input 34, display 36, and/or processor 33. In another embodiment, medical device 30, 32 is capable of stand-alone operation, and is further configured to function as electronic device 12, should communication with electronic device 12 be interrupted. In another embodiment, medical device 30, 32 may include processor, memory and communication capability, but does not have an input 34 or a display 36. In still another embodiment, the medical device 30, 32 may include an input 34, but lack a display 36.

In some embodiments, the system 10 may additionally include a remote devices 50, 52. The remote device 50, 52 may include a processor 53, which may be identical or similar to the processor 33 or processor 14, a memory or other data storage unit 54, a input device 56, which may include any one or more of the input devices described hereinabove, a display unit 58 which may include any one or more of the display units described hereinabove, and a communication I/O circuitry 60. The remote device 50, 52 may be configured to communicate with the electronic device 12 or medical devices(s) 30, 32 via any wired or wireless communication interface 62, which may include any of the communication interfaces or links described hereinabove. Although not specifically shown, remote device 50, 52 may also be configured to communicate directly with one or more medical devices 30, 32, instead of communicating with the medical device through electronic device 12.

System 10 may be provided in any of a variety of configurations, and examples of some such configurations will now be described. It will be understood, however, that the following examples are provided merely for illustrative purposes, and should not be considered limiting in any way. Those skilled in the art may recognize other possible implementations of a fully closed-loop, semi closed-loop, or open loop diabetes control arrangement, and any such other implementations are contemplated by this disclosure.

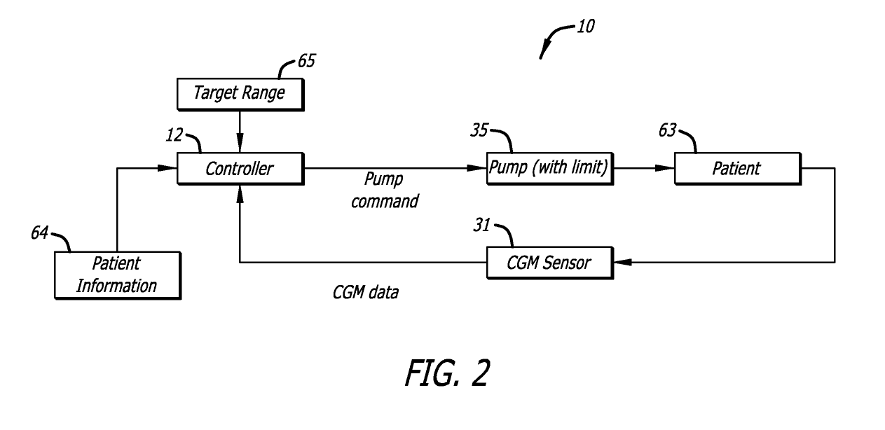

In a first exemplary implementation of the system 10, the medical device 30, 32 is provided in the form of one or more sensors 31 (FIG. 2) or sensing systems that are external to the user's body and/or sensor techniques for providing information relating to the physiological condition of the user. Examples of such sensors or sensing systems may include, but should not be limited to, a glucose strip sensor/meter, a body temperature sensor, a blood pressure sensor, a heart rate sensor, one or more bio-markers configured to capture one or more physiological states of the body, for example, HBA1C, or the like. In implementations that include a glucose sensor, system 10 may be a fully closed-loop system operable in a manner to automatically monitor glucose and deliver insulin, as appropriate, to maintain glucose at desired levels. Information provided by any such sensors and/or sensor techniques may be communicated by system 10 using any one or more wired or wireless communication techniques.

The various medical devices 30, 32 may additionally include an insulin pump 35 (FIG. 2) configured to be worn externally to the user's body and also configured to controllably deliver insulin to the user's body. In one such embodiment, medical devices 30, 32 include at least one implantable or externally worn drug pump. In one embodiment, an insulin pump is configured to controllably deliver insulin to the user's body. In this embodiment, the insulin pump is also configured to wirelessly transmit information relating to insulin delivery to the handheld device 12. The handheld device 12 is configured to monitor insulin delivery by the pump, and may further be configured to determine and recommend insulin bolus amounts, carbohydrate intake, exercise, and the like to the user. The system 10 may be configured in this embodiment to provide for transmission of wireless information from the handheld device 12 to the insulin pump.

In an implementation of the system 10, the electronic device 12 is provided in the form of a handheld device, such as a PDA or other handheld device. In a further embodiment, the handheld device 12 is configured to control insulin delivery to the user by determining insulin delivery commands and transmitting such commands to an insulin pump 35 (FIG. 2). The insulin pump, in turn, is configured to receive the insulin delivery commands from the handheld device 12, and to deliver insulin to the user according to the commands. The insulin pump, in this embodiment, may further process the insulin pump commands provided by the handheld unit 12 The system 10 will typically be configured in this embodiment to provide for transmission of wireless information from the insulin pump back to the handheld device 12 to thereby allow for monitoring of pump operation. The system 10 may further include one or more implanted and/or external sensors of the type described in the previous example.

Those skilled in the art will recognize other possible implementations of a fully closed-loop, semi closed-loop, or open loop diabetes control arrangement using at least some of the components of the system 10 illustrated in FIG. 1. For example, the electronic device 12 in one or more of the above examples may be provided in the form of a PDA, laptop, notebook or personal computer configured to communicate with one or more of the medical devices 30, 32, at least one of which is an insulin delivery system, to monitor and/or control the delivery of insulin to the user. In further embodiments, electronic device may include a communication port 22 in the form of a BLUETOOTH or other wireless transmitter/receiver, serial port or USB port, or other custom configured serial data communication port. In some embodiments, remote device 50, 52 is configured to communicate with the electronic device 12 and/or one or more of the medical devices 30, 32, to control and/or monitor insulin delivery to the user, and/or to transfer one or more software programs and/or data to the electronic device 12. Remote device 50, 52 may take the form of a PC, PDA, laptop or notebook computer, handheld or otherwise portable device, and may reside in a caregiver's office or other remote location. In the various embodiments, communication between the remote device and any component of the system 10 may be accomplished via an intranet, internet (for example, world-wide-web), cellular, telephone modem, RF, USB connection cable, or other communication link 62. Any one or more internet protocols may be used in such communications. Additionally, any mobile content delivery system; for example, Wi-Fi, WiMAX, BLUETOOTH, short message system (SMS), or other message scheme may be used to provide for communication between devices comprising the system 10.

FIG. 2 illustrates the components, and operation and control flow, of a closed-loop system. In the depicted embodiment, the system generally includes a sensor and a pump, and a controller module for receiving input from the sensor and for controlling the pump. The term “controller module” as used herein is defined as a hardware device that receives a signal representative of a glucose (for example, from a sensor) and produces signals to control an insulin delivery device (for example, a pump). In some embodiments, the controller module is part of or includes electronic device 12. In some embodiments, electronic device 12 (or handheld controller 12) is part of or includes the controller module. Thus, the controller module may be depicted in the drawings as either a controller or an electronic device 12, and the terms handheld electronic device, controller module, electronic device, and handheld device, are used herein interchangeably. In some embodiments, the controller module is hardware included with or interconnected to electronic device 12. In further embodiments, the controller module is hardware included with or interconnected to sensor 31 and/or pump 35.

In some embodiments, the sensor and/or pump is part of, or includes medical device 30, 32 (that is, medical device 30, 32 can be a pump or a sensor). In some embodiments, the controller module may be part of, or be integrated with, a sensor 31 or a pump 35, or other medical devices 30, 32. Handheld controller 12 preferably has a user interface screen 20 to display information to the user and to request from the user the input of parameters and/or commands. Handheld controller 12 may further comprise a processor 14, and an input means 18, such as buttons or a touch screen, for the user to input and/or set parameters and commands to the system.

Handheld controller 12 includes a memory means 16 configured to store parameters and one or more algorithms that may be executed by processor 14. For example, memory means 16 may store one or more predetermined parameters or algorithms to evaluate glucose data, trends in that data, and future prediction models. A user may also input parameters using input 18 to provide user-specific algorithms such as pumping patterns or algorithms for determining an amount of drug (that is, insulin) to be delivered by an insulin delivery device (IDD), pump 35. Input 18 may also be used to send commands or to bring up a menu of commands for the user to choose from. In some embodiments, these components (that is, input, processor, and memory) comprise the control module. The information may be displayed, for example, on display 20 of handheld controller 12, and user input may be received via input 18. In one embodiment, handheld controller 12 takes into account for both deliveries commanded by the controller as well as deliveries commanded by human input intended to correct or compensate for specific aspects not necessarily known to the controller. The components of the embodiments may cooperatively work together as a single device or separate physical devices.

In one embodiment, handheld controller 12 is provided to allow the user to view via graphical display 20 his or her glucose levels and/or trends and to control the pump 35. Handheld controller 12 sends commands to operate pump 35, such as an automatic insulin basal rate or bolus amount. Handheld controller 12 may automatically send commands based on input from sensor 31 or may send commands after receiving user input via input 18 or input 34 on medical device 30, 32. In at least one embodiment, handheld controller 12 analyzes data from sensor 31 and/or pump 35, and/or communicates data and commands to them. In one embodiment, handheld controller 12 automatically sends the commands to pump 35 based on a sensor reading. Handheld controller 12 may also send commands to direct the pumping action of the pump 35. Handheld controller 12 sends and receives data to and from sensor 31 over a over a wired connection or wireless communication protocol 42. In another embodiment, data based on the reading is first provided to handheld controller 12 which analyzes the data and presents information to a user or a health care provider (for example, using remote device 50, 52), wherein human input is required to generate the command. For example, handheld controller 12 may request an acknowledgment or feedback from the user before sending the commands, allowing the user to intervene in command selection or transmission. In a further embodiment, handheld controller 12 merely sends alerts or warnings to the user and allows the user to manually select and send the commands via the input 18 of handheld controller 12. In yet another embodiment handheld controller 12 manages commands originated by the control algorithm with or without user approval or intervention, and commands initiated by the user are independent of the control algorithm. The purpose of handheld controller 12 is to process sensor data in real-time and determine whether the glucose levels in a user is too high or too low, and to provide a prediction of future glucose levels based upon sensor readings and the current basal rate and/or recent bolus injections.

In some embodiments, handheld controller 12 includes a means for calibrating the system, including, inputting at the device a finger stick glucose measurement or taking an actual blood sample to obtain a glucose measurement. The device may be integrated with a strip port so that a user may use the strip port to take a manual glucose reading. The strip port includes a known calibration and is configured to take a blood reading to provide a value representative of a glucose. The reading provided from the strip port is internally received at handheld controller 12 and compared to a value from sensor 31 to configure and/or calibrate the system.

Sensor 31 is configured to read a glucose level of a user and to send the reading to be analyzed by handheld controller 12. In some embodiments, sensor 31 is a glucose monitor with a strip port for manually receiving a blood sample. In the depicted embodiments, sensor 31 is a continuous glucose monitoring (CGM) sensor that pierces and/or is held in place at the surface of a user's skin to continuously monitor glucose levels in a user. In an embodiment, CGM sensor 31 (a portable medical device 30, 32) is attached to the surface of a user's skin and includes a small sensor device that at least partially pierces the user's skin and is located in the dermis to be in contact the interstitial fluid. The sensor device may also be held in place at the skin by a flexible patch. Accordingly, CGM Sensor 31 may provide continuous monitoring of user glucose levels. The analyte monitoring system may also include a transmitter and/or receiver for transmitting sensor data to a separate device (for example, pump 35 or handheld electronic device 12). In some embodiments, CGM sensor 31 is in the form of a skin-mounted unit on a user's arm.

In the depicted embodiments, an insulin device or pump 35 delivers insulin to the user through a small tube and cannula (also known as the “infusion set”) percutaneously inserted into the user's body. Insulin pump 35 may be in the form of a medical pump, a small portable device (similar to a pager) worn on a belt or placed in a pocket, or it may be in the form of a patch pump that is affixed to the user's skin. In one embodiment, pump 35 is attached to the body by an adhesive patch and is normally worn under clothes. Pump 35 is preferably worn on the skin, includes a power supply, and is relatively small and of a low profile so that it can be hidden from view in a pocket or attached to the skin under clothing.

The pump has disposable and non-disposable components. The disposable components include the reservoir and cannula and (optional) adhesive patch. The non-disposable/reusable component includes the pumping electronics, transmitter and/or receiver, and pump mechanics (not shown). Pump 35 and cannula may be part of the same physical device or comprise separate modules. Pump 35 may also comprise a transmitter and/or receiver for transmitting and/or receiving a signal via connection 42 from handheld controller 12 so that it can be controlled remotely and can report pump-specific data to a remote location.

When provided as an integrated system, the components of system 10 work together to provide real-time continuous glucose monitoring and control of an insulin pump and to allow a user to take immediate corrective or preventative action when glucose levels are either too high or too low. Because pump 35 and sensor 31 are miniaturized they may have very limited control panels, if any at all, and thus, in some embodiments, sensor 31, pump 35, and controller 12 may all be integrated into a single device. In other embodiments, sensor 31, pump 35, and controller 12 may be organized as two or three separate components. The components may be in wired communication, radio communication, fluid connection, or other communication protocol suitable for sending and receiving information between the components. Some components may be constructed to be reusable while others are disposable. For example, the cannula and the sensor may be disposable pieces apart from the pump 35 and CGM sensor 31 which are both preferably reusable. The cannula and/or sensor will preferably be in fluid isolation from other components. Each component may have modular fittings so that the disposable components may interact with the non-disposable components while remaining in fluid isolation from each other.

Generally, the concentration of glucose in a person changes as a result of one or more external influences such as meals and exercise, and also changes resulting from various physiological mechanisms such as stress, illness, menstrual cycle and the like. In a person with diabetes, such changes can necessitate monitoring the person's glucose level and administering insulin or other glucose-altering drug, for example, glucose lowering or raising drug, as needed to maintain the person's glucose within desired ranges. In any of the above examples, the system 10 is thus configured to determine, based on some amount of user-specific information, an appropriate amount, type and/or timing of insulin or other glucose-altering drug to administer in order to maintain normal glucose levels without causing hypoglycemia or hyperglycemia.

In some embodiments, the system 10 is configured in a manner to control one or more external (for example, subcutaneous, transcutaneous or transdermal) and/or implanted insulin pumps to automatically infuse or otherwise supply the appropriate amount and type of insulin to the user's body in the form of one or more insulin boluses. Such insulin bolus administration information may be or include, for example, insulin bolus quantity or quantities, bolus type, insulin bolus delivery time, times or intervals (for example, single delivery, multiple discrete deliveries, continuous delivery, etc.), and the like. Examples of user supplied information may be, for example but not limited to, user glucose concentration, information relating to a meal or snack that has been ingested, is being ingested, or is to be ingested sometime in the future, user exercise information, user stress information, user illness information, information relating to the user's menstrual cycle, and the like.

System 10 may also include a delivery mechanism for delivering controlled amounts of a drug; for example, insulin, glucagon, incretin, or the like to pump 35, and/or offering an actionable therapy recommendation to the user via the display 20, for example, ingesting carbohydrates, exercising, etc. In other embodiments, the system 10 is configured in a manner to display or otherwise notify the user of the appropriate amount, type, and/or timing of insulin in the form of an insulin recommendation. In such embodiments, hardware and/or software forming part of the system 10 allows the user to accept the recommended insulin amount, type, and/or timing, or to reject it. If accepted, the system 10, in one embodiment, automatically infuses or otherwise provides the appropriate amount and type of insulin to the user's body in the form of one or more insulin boluses. If, on the other hand, the user rejects the insulin recommendation, hardware and/or software forming part of the system 10 allows the user to override the system 10 and manually enter insulin bolus quantity, type, and/or timing. The system 10 is then configured in a manner to automatically infuse or otherwise provide the user specified amount, type, and/or timing of insulin to the user's body in the form of one or more insulin boluses.

The appropriate amount and type of insulin corresponding to the insulin recommendation displayed by system 10 may be manually injected into, or otherwise administered to, the user's body. It will be understood, however, that the system 10 may additionally be configured in like manner to determine, recommend, and/or deliver other types of medication to a user.

System 10 is operable to determine and either recommend or administer an appropriate amount of insulin or other glucose lowering drug to the user in the form of one or more insulin boluses. In determining such appropriate amounts of insulin, system 10 requires at least some information relating to one or more external influences and/or various physiological mechanisms associated with the user. For example, if the user is about to ingest, is ingesting, or has recently ingested, a meal or snack, the system 10 generally requires some information relating to the meal or snack to determine an appropriate amount, type and/or timing of one or more meal compensation boluses. When a person ingests food in the form of a meal or snack, the person's body reacts by absorbing glucose from the meal or snack over time. For purposes of this disclosure, any ingesting of food may be referred to hereinafter as a “meal,” and the term “meal” therefore encompasses traditional meals, for example, breakfast, lunch and dinner, as well as intermediate snacks, drinks, etc.

Referring to FIG. 2, in some embodiments, in order for continuous glucose monitoring and/or control system 10, including controller 12, sensor 31, and pump 35, to be most effective in treating a user 63, a user information profile 64 and optimal control parameters 65 are provided. In one embodiment, certain control parameters, for example, target glucose threshold, an overall glucose safe range, and the like can often be predetermined based on known values for common user types and are typically known in the art. Other embodiments, may supplement known ranges by information observed and determined by user's 63 health care provider (HCP). In some embodiments, user information profile includes information specific to patent 63, including a quantified glucose absorption profile created based on, for example, body type, race, known tolerances, historical data and the like.

The general shape of a glucose absorption profile for any person rises following ingestion of the meal, peaks at some measurable time following the meal, and then decreases thereafter. The speed, that is, the rate from beginning to completion, of any one glucose absorption profile typically varies for a person by meal composition, by meal type or time (for example, breakfast, lunch, dinner, or snack) and/or according to one or more other factors, and may also vary from day-to-day under otherwise identical meal circumstances. Generally, the information relating to such meal intake information supplied by the user to the system 10 should contain, either explicitly or implicitly, an estimate of the carbohydrate content of the meal or snack, corresponding to the amount of carbohydrates that the user is about to ingest, is ingesting, or has recently ingested, as well as an estimate of the speed of overall glucose absorption from the meal by the user.

The estimate of the amount of carbohydrates that the user is about to ingest, is ingesting, or has recently ingested, may be provided by the user in any of various forms. Examples include, but are not limited to, a direct estimate of carbohydrate weight (for example, in units of grams or other convenient weight measure), an amount of carbohydrates relative to a reference amount (for example, dimensionless), an estimate of meal or snack size (for example, dimensionless), and an estimate of meal or snack size relative to a reference meal or snack size (for example, dimensionless). Other forms of providing for user input of carbohydrate content of a meal or snack will occur to those skilled in the art, and any such other forms are contemplated by this disclosure.

The estimate of the speed of overall glucose absorption from the meal by the user may likewise be provided by the user in any of various forms. For example, for a specified value of the expected speed of overall glucose absorption, the glucose absorption profile captures the speed of the meal taken by the user. As another example, the speed of overall glucose absorption from the meal by the user also includes time duration between ingesting of the meal by a person and the peak glucose absorption of the meal by that person, which captures the duration of the meal taken by the user. The speed of overall glucose absorption may thus be expressed in the form of meal speed or duration. Examples of the expected speed of overall glucose absorption parameter in this case may include, but are not limited to, a compound parameter corresponding to an estimate of the meal speed or duration (for example, units of time), a compound parameter corresponding to meal speed or duration relative to a reference meal speed or duration (for example, dimensionless), or the like.

As another example of providing the estimate of the expected speed of overall glucose absorption parameter, the shape and duration of the glucose absorption profile may be mapped to the composition of the meal. Examples of the expected speed of overall glucose absorption parameter in this case may include, but are not limited to, an estimate of fat amount, protein amount and carbohydrate amount (for example, in units of grams) in conjunction with a carbohydrate content estimate in the form of meal size or relative meal size, an estimate of fat amount, protein amount and carbohydrate amount relative to reference fat, protein and carbohydrate amounts in conjunction with a carbohydrate content estimate in the form of meal size or relative meal size, and an estimate of a total glycemic index of the meal or snack (for example, dimensionless). The term “total glycemic index” is defined for purposes of this disclosure as a parameter that ranks meals and snacks by the speed at which the meals or snacks cause the person's blood sugar to rise. Thus, for example, a meal or snack having a low glycemic index produces a gradual rise in blood sugar whereas a meal or snack having a high glycemic index produces a fast rise in blood sugar. One exemplary measure of total glycemic index may be, but is not limited to, the ratio of carbohydrates absorbed from the meal and a reference value, for example, derived from pure sugar or white bread, over a specified time period, for example, 2 hours. Other forms of providing for user input of the expected overall speed of glucose absorption from the meal by the user, and/or for providing for user input of the expected shape and duration of the glucose absorption profile generally will occur to those skilled in the art, and any such other forms are contemplated by this disclosure.

Generally, the concentration of glucose in a person with diabetes changes as a result of one or more external influences such as meals and/or exercise, and may also change resulting from various physiological mechanisms such as stress, menstrual cycle and/or illness. In any of the above examples, the system 10 responds to the measured glucose by determining the appropriate amount of insulin to administer in order to maintain normal glucose levels without causing hypoglycemia. In some embodiments, the system 10 is implemented as a discrete system with an appropriate sampling rate, which may be periodic, aperiodic or triggered, although other continuous systems or hybrid systems may be implemented as described above.

As one example of a diabetes control system, one or more software algorithms may include a collection of rule sets which use (1) glucose information, (2) insulin delivery information, and/or (3) subject inputs such as meal intake, exercise, stress, illness and/or other physiological properties to provide therapy, etc., to manage the user's glucose level. The rule sets are generally based on observations and clinical practices as well as mathematical models derived through or based on analysis of physiological mechanisms obtained from clinical studies. In the exemplary system, models of insulin pharmacokinetics and pharmacodynamics, glucose pharmacodynamics, meal absorption and exercise responses of individual users are used to determine the timing and the amount of insulin to be delivered. A learning module may be provided to allow adjustment of the model parameters when the user's overall performance metric degrades (for example, adaptive algorithms, using Bayesian estimates, may be implemented). An analysis model may also be incorporated which oversees the learning to accept or reject learning. Adjustments are achieved utilizing heuristics, rules, formulae, minimization of cost function(s) or tables (for example, gain scheduling).

Predictive models can be programmed into the processors of the system using appropriate embedded or inputted software to predict the outcome of adding a controlled amount of insulin or other drug to a user in terms of the an expected glucose value. The structures and parameters of the models define the anticipated behavior.

Any of a variety of controller design methodologies, such as PID systems, full state feedback systems with state estimators, output feedback systems, LQG controllers, LQR controllers, eigenvalue/eigenstructure controller systems, and the like, could be used to design algorithms to perform physiological control. They typically function by using information derived from physiological measurements and/or user inputs to determine the appropriate control action to use. While the simpler forms of such controllers use fixed parameters (and therefore rules) for computing the magnitude of control action, the parameters in more sophisticated forms of such controllers may use one or more dynamic parameters. In some embodiments, the one or more dynamic parameters take the form of one or more continuously or discretely adjustable gain values. In some embodiments, specific rules for adjusting such gains are defined on an individual basis, and, in other embodiments, on the basis of a user population. In either case these rules will typically be derived according to one or more mathematical models. Such gains are scheduled according to one or more rule sets designed to cover the expected operating ranges in which operation is typically nonlinear and variable, thereby reducing sources of error.

Model based control systems, such as those utilizing model predictive control algorithms, can be constructed as a black box wherein equations and parameters have no strict analogs in physiology. Rather, such models may instead be representations that are adequate for the purpose of physiological control. The parameters are typically determined from measurements of physiological parameters such as glucose, insulin concentration, and the like, and from physiological inputs such as food intake, alcohol intake, insulin doses, and the like, and also from physiological states such as stress level, exercise intensity and duration, menstrual cycle phase, and the like. These models are used to estimate current glucose or to predict future glucose values. Such models may also take into account unused insulin remaining in the blood after a bolus is given, for example, in anticipation of a meal. Such unused insulin will be variously described as unused, remaining, or “insulin on board.”

Insulin therapy is derived by the system based on the model's ability to predict glucose for various inputs. Other modeling techniques may be additionally used including for example, but not limited to, building models from first principles.

As described above, system 10 includes an analyte monitor that continuously monitors the glucose levels in a user. The controller module is programmed with appropriate software and uses models as described above to predict the effect of carbohydrate ingestion and exercise, among other factors on the predicted level of glucose. Such a model must also take into account the amount of insulin remaining in the blood stream from a previous bolus or basal rate infusion when determining what or whether or not to provide a bolus of insulin.

The controller module is typically programmed to provide a “basal rate,” which is the rate of continuous supply of insulin by an insulin delivery device such as a pump that is used to maintain a desired glucose level in the bloodstream of a user. Periodically, due to various events that affect the metabolism of a user, such as eating a meal or engaging in exercise, a “bolus” is required. A “bolus” is a specific amount of insulin that is required to raise the blood concentration of insulin to an effective level to counteract the effects of the ingestion of carbohydrates during a meal and also takes into account the effect of exercise on the glucose level.

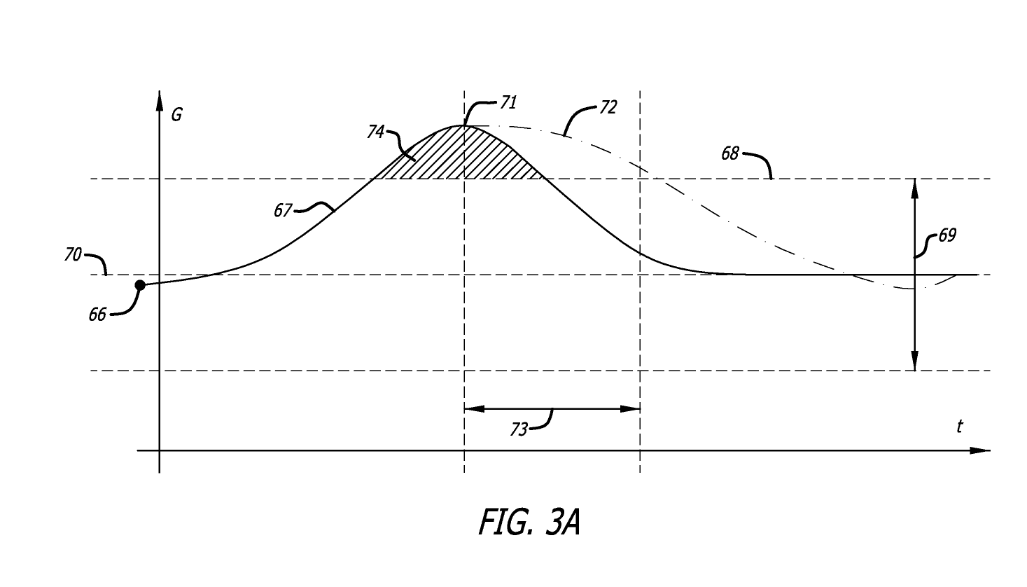

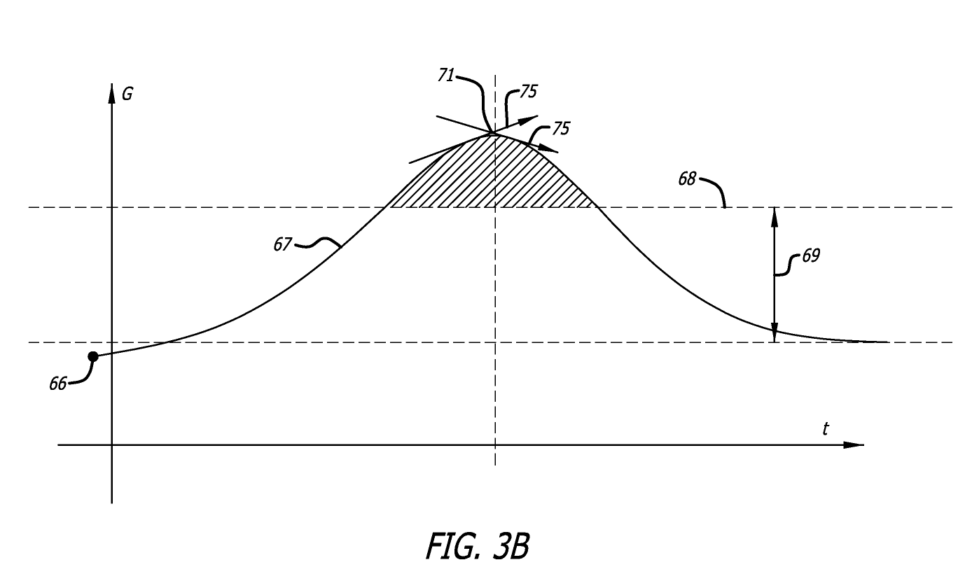

A proactive “glycemia exposure avoidance” system in accordance with aspects of the present invention can further enhance the efficacy and usability of a combined and/or integrated CGM and pump device for the user. As such, the present system incorporates a post-prandial peak driven glycemia exposure avoidance in a combined and/or integrated CGM and pump system. An aspect of the invention is to reduce the overall exposure of a user to a high glucose. Referring to FIG. 3A, after a meal 66, and at a certain time t, a user's glucose value 67 will trend in a positive direction. System 10 is programmed with a high (or low) target glucose threshold 68, an overall glucose safe range 69 below (or above) threshold 68, and a target glucose level 70 that serves as a guide for calculating an amount of insulin or carbohydrate necessary to maintain glucose values within safe range 69. Even if alerted by an alarm prior to or at threshold 68 and a correction bolus is given, the precise point 71 at which the glucose value 67 begins to trend downward will remain unknown and often there will be a period of time in which glucose value 67 will remain outside safe range 69. Studies show that, even after administration of a bolus, the peak can occur between 1 and 2 hours after a meal. Without careful manual monitoring of the glucose value (which, if self-checked by standard means can also be painful) this post prandial peak 71 can go unnoticed and subject the user to a delayed decrease 72 in insulin and thus an increased glucose exposure for an unnecessary and sometimes dangerous length of time. System 10 solves this problem by identifying and detecting the post prandial peak 71 at the maximum glucose value after a food & correction bolus is initiated and provides the user with the appropriate information to reduce insulin action time 73 to minimize the user's high glucose exposure 74.

In one embodiment, using system 10, the user provides an input at input 18 of electronic device 12 (or input 34 of medical device 30, 32) at the time of performing a food & correction bolus to set up a reminder when the system 10 is to act to detect a peak after the meal, for example, from the CGM data received at controller 12 from medical device 30, 32. In some embodiments, projected alarms settings can be further set and refined by using insulin-on-board (IOB) and meal information, and provide information to a user/user of carbohydrate deficit state, insulin deficit state, and low blood-glucose management.

With reference to FIG. 3B, in one embodiment, an algorithm detects the post prandial peak at the maximum glucose 71 value after a food & correction bolus is initiated and the trend arrows 75 begin to trend downward. In one embodiment, a moving average or smoothing algorithm is also used to reduce noise artifacts, thus providing an accurate detection of the peak.

Consider electronic device 12 (including a display with trend arrows) and pump 35 at the time a meal event 66 is indicated, including a “food” bolus of some sort using a bolus calculator tool (food bolus, food and correction bolus, etc.). In some embodiments, system 10 delays detection after a food ingestion to ensure that the food is indeed being digested and the glucose value 65 is rising before starting the peak detection process. In one embodiment, this delay is based on a fixed time interval (for example, after thirty minutes), a number of consecutive rises in glucose readings (for example, two consecutive ten minute readings are rising), and time to reach a certain minimum glucose rate increase (for example, 5 mg/dL per minute). Determining this delay time may also require examining population data looking at glucose response to the food event. In one embodiment, system 10 incorporates in the dataset different pre-prandial glucose trends (for example, those that have rapid fluctuation before the meal). In another embodiment, the system 10 may be set to start peak detection when a certain glucose level is reached, or a series of values indicate a trend matching a predetermined alert profile.

In one embodiment, controller 12 receives a set of readings from CGM sensor 31 and records them on memory 16. Sampling time can be based on the CGM sensor sampling rate or, in some embodiments, set by the user at input 18 of controller 12. In one embodiment, the sampling time is set to 1 sample per minute. Subsequent sets of readings are made and stored. The number of readings for each set may be variable, and may be, in some cases, set to as low as two consecutive readings. Sets of readings may also overlap such that, for example, a first reading in a set and a second reading in a set may also be readings of prior and subsequent sets. As the readings are sampled, a determination is made by the processor 14 of a current trend in glucose levels in the user. For the purpose of this disclosure, the term “trend” is used in the geometric sense to indicate a general direction, either upward or downward, for the slope of a curve. A curve may signify an amount of glucose over time.

A change in trend may indicate a peak or valley in glucose level. For the purpose of this disclosure, the term “vertex” is used in the geometric sense; that is, it is a point of where the first derivative or slope of curvature is zero. It therefore can be a peak or a valley of a curve. The trend may then be displayed on the display 12 as a trend arrow or other graphical indication indicating the trend in glucose. In some embodiments, when the trend changes, controller 12 is configured to send a signal or alert to other components of the system. In one embodiment controller 12 may signal the user via display 20 and a speaker alert. In one embodiment, processor 14 may signal sensor 31 or pump 35 for further action. For example, pump 35 may take up remedial measures to deactivate or adjust drug delivery. In some embodiments, in which the vertex is a peak, controller 61 and/or a processor associated therewith is configured to perform an action to calculate a dose of medication sufficient to reduce the glucose level more rapidly to the target glucose level within the safe range and to decrease an amount of high glucose exposure than if the action was not taken. In some embodiments, in which the vertex is a valley, the action calculates an amount of carbohydrates sufficient to increase the glucose level more rapidly to the target glucose level. In further embodiments, the action includes calculating a reduction in a basal rate of a medication administration sufficient to raise the glucose level more rapidly to the target glucose level.

In one embodiment, the system employs the following steps to determine a peak in glucose:

1) Determine the peak by tracking post-prandial glucose trend arrows 75 (FIG. 3B) and the time t when the increasing arrow turns to decreasing arrow is the time to indicate to the user that post-prandial peak 71 is reached.

2) Determine the peak by numeric calculation of the rate of change of glucose and see when this calculated rate changes sign. A typical rate can be calculated by a simple calculation of change in glucose divided by change in time. For example, a rate can be calculated between two time points t₀ and t₁ by the equation:

rate=[Glucose(t₁)−Glucose(t₀)]/t₁−t₀

If the glucose is increasing, then rate is>0. If glucose is decreasing, then rate is<0. (The rate calculation underlying the presentation of the rate arrows may be used instead of additional calculation, essentially being the same as step 1.) In one embodiment the calculation of this rate is done every minute, or at whatever the current frequency of glucose reading is for the system, to provide earliest possible indication of peak.

3) Remove noise artifacts from the glucose data by a smoothing algorithm to smooth out the glucose signal before the rate calculation to reduce false positives (identifying a peak when it is really not a peak). In some embodiments, trend line smoothing is generally over n minutes. In one embodiment, for example, smoothing occurs over a time period of 3 to 30 minutes. In one embodiment, an exponential smoothing algorithm may be employed to smooth each glucose value before the rate calculation. For every glucose (G) at time tᵢ, a smoothed glucose (SG) value can be obtained according to:

SG(tᵢ)=alpha*Glucose(tᵢ)+(1-alpha)*SG(tᵢ₋₁)

where alpha is the parameter controlling the smoothing, and is constrained between zero and one, 0<alpha<=1.

Given that it is expected there to be a definite trend, the smoothed values will tend to lag behind the unsmoothed glucose data in this simple exponential smoothing scheme, unless alpha is chosen to be close to 1.

In one embodiment, double exponential smoothing is used to avoid the time delay (if the delay becomes great enough) by introducing another “trend” variable (essentially a rate calculation) as a component of smoothing. Such double exponential smoothing can be employed to smooth each glucose value before the rate calculation. For every glucose (G) at time you can obtain a smoothed glucose (SG) value:

SG(tᵢ)=alpha*Glucose(tᵢ)+(1-alpha)*(SG(tᵢ₋₁)+Trend(tᵢ₋₁)

Trend (tᵢ)=beta*(SG(tᵢ)−SG(tᵢ₋₁))+(1-beta)*Trend(tᵢ₋₁)

where alpha is the parameter controlling the smoothing of glucose, and beta is the parameter controlling the smoothing of the trend, and both are constrained between zero and one, 0<alpha, beta<=1. Some optimization will be needed to find best alpha and beta values.

Once the peak is reached, the system can notify the user to activate the high glucose avoidance analysis. In one embodiment, the avoidance strategy requires Insulin Action Time, Insulin On-board (IOB), and a user's insulin sensitivity (ISF). An analysis process can be described using the following user exemplary settings:
Target Threshold: 110 mg/dL
High Alarm Threshold: 240 mg/dL
Low Alarm Threshold: 70 mg/dL
Carb Ratio: IU for 15 grams of carb
Insulin Sensitivity Factor (ISF): 1 Units for 50 mg/dL
Insulin Action Time: 5 hours
Current CGM: 230 mg/dL
Current IOB: 1.1 units
   In one embodiment, Current glucose−(IOB*ISF)=future glucose after Insulin Action Time. For example, 230 mg/dL−(1.1 units*50 mg/dL/unit)=230 mg/dL−55 mg/dL=175 mg/dL. Thus, using the above parameters, calculations show that in five hours, the user will lower his or her glucose by 55 mg/dL (IOB*ISF) and be around 175 mg/dL.

4) Without triggering a high alarm, system 10 recommends to the user to self-administer a little more insulin (X units). In one embodiment, Additional Insulin needed={[Future glucose after Insulin Action Time]-Target Threshold}/ISF. For example, (175 mg/dL (from the calculation before)−110 mg/dL)/50 mg/dL/unit=65/50=1.3 units of insulin. Note: with a passive the system, the user may not choose to act based on the fact that IOB is zero—for fear of stacking insulin or wanted to wait if this is the peak or not.

The benefit of the present invention is to provide the user a means to proactively manage their glucose after a meal. This allows the user to detect and counter “insulin deficit” situation much earlier and prevent an extended downward trend 70 (FIG. 3A) in user glucose.

In some embodiments, during the analysis in step 3, above, the user could be in three possible states:

Case A) OK state;

Case B) Carb Deficit State;

Case C) Insulin Deficit state (as described above).

It is also possible that the user could be in a carbohydrate deficit state already, with the peak detection and avoidance analysis. In one embodiment, system 10 incorporates the Carb Ratio (for example, IU) to facilitate future basal insulin reduction if user is in B) Carb Deficit State.

For example, using the same user setting as earlier:
Current CGM: 230 mg/dL
Current IOB: 3.1 units
   The above calculation shows that in five hours, the user's glucose will lower by 155 mg/dL (IOB*ISF) and be around 75 mg/dL. This is way below the target, and close to the low alarm, so this state is categorized as a Carb Deficit State. In some aspects, this is the result of over-dosing of insulin in response to an earlier meal. In some aspects, this situation might not trigger a low alarm within five hours.

In one embodiment, once the device knows that the user is in carbohydrate-deficit state, the system displays the following recommendations on graphical display 20:

a) Alert the user to eat X grams of carbs now or later to avoid the low. In some aspects, this is not a viable choice right now because the user just ate. In one embodiment, the system switches directly to the basal reduction choice.

b) Alert the user to reduce the basal rate by X units per hour in the next five hours (insulin action time) X=(Target-Future Level)/ISF/Insulin Action Time=(110−75)/50/5=0.14 units per hour. It should be noted that if the Future Level falls below the Low Alarm Threshold, then the Target parameter in the above equation can be set to the Low Alarm Threshold to provide a small reduction recommendation. The actual value of this parameter is typically preset in response to a consultation with the user's clinician.

In sum, without knowing exactly when post-prandial peak occurs, a user-user may not be able to intervene at the most optimal time. In one embodiment, system 10 uses CGM data to detect a post-prandial peak and reduce glycemic exposure. In one embodiment, an audible alarm may sound when the peak has been calculated. In one embodiment the current and future trend values and/or peak may be graphically displayed on one or more displays 20, 36, 58 of system 10. Display may be user-initiated or may, in some instances, be ongoing, such as a constant graph or small trend indicator located on the display screen. In one embodiment, on confirmed peak detection, the controller module automatically signals pump 35 automatically to reduce basal rate. In another embodiment, the controller module may make an initial determination of a reduced basal rate and present the adjusted flow rate to the user on graphical display 20 or display 36 of medical device 30, 32. In one embodiment, the user is prompted to confirm the adjusted rate via input 18 of electronic device 12. In another embodiment, the user is prompted to confirm the adjusted rate via input 34 of medical device 30, 32. This pre-emptive pump control modification process further enhances the usability of system 10. Accordingly, optimal post-prandial insulin adjustment intervention based on post-prandial peak detection is more efficient than a passive system that allows the user to act on their own without knowing the moment of peak.

In one aspect, the present invention provides for calculating and/or adjusting a glucose-related performance metric, such as insulin control parameters used in calculating an amount of glucose to be injected over time in a CGM system, based upon, for example, current performance settings, control actions, and/or sampled glucose values. In some embodiments, the performance metric includes an average glucose expected from a present time to a future time. In some embodiments, the performance metric includes a slope of the levels of glucose over a selected window of time. In other embodiments, a performance metric may include, but is not limited to, a level of glucose at a point in the future, a number and/or time a particular value is out of tolerance, a number and/or time of hypoglycemic or hyperglycemic events, and/or a number and/or amount of bolus corrections.

In one embodiment, optimal control parameters are determined by retrospectively analyzing a user's data relevant to diabetes history or profile data. The results of this analysis are graphically displayed to the user and/or HCP, for instance, via display on one or more displays 20, 36, 58 of system 10. System 10 may make certain recommendations for adjusting drug delivery, and/or automatically adjust the user's diabetes management profile. In one embodiment, system 10 makes a confirmation request from the user or HCP, allowing a confirmation input via one or more inputs 18, 34, 56 prior to changes being made. In addition, the recommendations may be modified via manual input by the user or HCP. In a further embodiment, instead of determining control parameters, optimal insulin pump settings are determined.

In one embodiment, the sensor 31 samples glucose levels in a user at one or more sample times t=k. The system considers a closed loop control whose core controller architecture computes an amount of insulin u(k) at every sample time based on one or more of the following: (1) the difference between the latest CGM and a target value, (2) the difference between the latest CGM rate of change and a target rate of change (which may be a function of CGM values and other information), and (3) the present amount of insulin on board (IOB) and/or target amount of IOB. In one embodiment, at any sample time k, a control action is described by:

u(k)=u_{g}(k)+uᵣ(k)−u_{IOB}(k)

u_{g}(k)=K_{g}×(G_{CGM}(k)−G_{target})

uᵣ(k)=Kᵣ×(Ġ_{CGM}(k)−Ġ_{target})

u_{IOB}(k)=K₁×IOB(k)

Wherein u(k) represents an amount of insulin given at time t=k, based on component values u_{g}, uᵣ, and u_{IOB}. Each component value is based, in part, on one or more control parameters (for example, scaling factors K_{g}, Kᵣ, K₁, and/or target values Ġ_{target}, and G_{target}) that, according to the disclosed embodiment, are adjusted over time. In one embodiment, component value u_{g}, representing an amount of insulin given proportional to a sensed glucose G_{CGM} at time k and the target glucose G_{target}, is adjusted by control parameter K_{g}. In one embodiment, K_{g} is initially set to a value determined by the user's Insulin Scaling Factor (ISF) distributed over a number of samples over a meal duration. An aspect of the invention is to automatically adjust the control parameters to accommodate real-time circumstances and situations rather than relying on the estimation of the parameters of a hypothetical model, in order to derive the best control parameters, where the parameters may only be manually changed periodically by a user or the user's HCP. In some embodiments, in the case of insulin delivery, a negative u(k) value at any sample time k implies no delivery. In some embodiments, repeated negative values over a duration exceeding a certain pre determined threshold is a strong indicator of an insulin stack up that will likely lead to hypoglycemia. In one embodiment, such an event will trigger an alarm alerting the user to either confirm a glucose reading and/or delivery and/or take rescue carbs.

In one embodiment, correction values uᵣ and, u_{IOB} are adjusted in part on correction scaling factors Kᵣ, and, K_{IOB}, respectfully. Control parameter Kᵣ is adjusted relative to glucose rate of change over time (initialized to=0) and control parameter K₁ is adjusted relative to an amount of insulin remaining in the body over time (initialized to=1 and may be adjusted downward if IOB consistently remains high after meals). In further embodiments, system 10 also calculates and adjusts the glucose target G_{target} and the glucose target rate of change Ġ_{target} with respect to any time k. In one instance, Ġ_{target} and G_{target} may remain constant, and, adjusted slowly if too many hypo events reveal that the current settings are found to be too aggressive.

In one embodiment, relevant historical data such as CGM values, CGM rates, glucose values, insulin delivery history, and recorded events such as meals, are received from sensor 31 and stored in memory 16 of controller 12. In some embodiments, where electronic device 12 is omitted, or controller module is integrated with medical device 30, 32, the values may be stored in memory 40 of medical device 30, 32.

In some embodiments, a series of performance metrics are computed based on the stored historical data. In one embodiment, data is collected from one window of time to another, and an adaptation rule is employed to determine a more suitable set of controller parameters K_{g}, Kᵣ, K₁, Ġ_{target} and G_{target} in real-time. In one embodiment, the window moves with time, discarding the oldest information in favor of new information. In another embodiment, the window jumps with time, in which the controller parameters remain fixed until a window with a completely new set of information becomes available.

In one embodiment, system 10 calculates the parameter adaptation of K_{g}, using a set of data including no appreciable CGM rate nor IOB. It is thus possible to use a nominal transfer function (that does not depend on a specific user) to scale the contribution of u_{g}, into values related to a performance metric. For instance, in one embodiment, a future performance metric is determined to be the average glucose expected from present to 30 minutes ahead. Let Y_{ahead30actual} be this value:

y_{target}_{_{30actual}}(k,K_{g})=ƒ(u_{g}(i|i=k−∞, . . . , k−0))

Let y_{ahead30desired} be the desired value. Then, the error incurred (at time k) is simply:

e_{ahead30}(k,K_{g})=y_{ahead30actual}(k,K_{g})−y_{ahead30desired}(k)

Thus, using the MIT Rule, a cost function for the adaptation of K_{g}, becomes:

$$J ( k , {K}_{g} ) = \frac{1}{2} {e}_{ahead 30}^{2} ( k , {K}_{g} )$$

Based on the current performance settings, and sampled control actions and glucose data collected by system 10, it becomes possible to adapt control parameter K_{g}. The update rule for K_{g}, is computed using the past data, and, using the MIT Rule as an example of a parameter adaptation method, the change in K_{g} is governed by:

$$\begin{matrix}\frac{d}{d t} {K}_{g} = - γ \frac{\partial }{\partial {K}_{g}} J ( k , {K}_{g} ) \\ = - γ {e}_{ahead 30}^{2} ( k , {K}_{g} ) \frac{\partial }{\partial {K}_{g}} [ {e}_{ahead 30}^{2} ( k , {K}_{g} ) ]\end{matrix}$$

Other parameter adaptation methods can be used without deviation from the scope of the present invention. For example, instead of taking the square of the error, absolute error can be defined as the cost function:

J(k,K_{g})=|e_{ahead30}(k,K_{g})|

Which results in the following parameter adaptation rule:

$$\begin{matrix}\frac{d}{d t} {K}_{g} = - γ \frac{\partial }{\partial {K}_{g}} J ( k , {K}_{g} ) \\ = - γsign ( {e}_{ahead 30} ( k , {K}_{g} ) ) \frac{\partial }{\partial {K}_{g}} [ {e}_{ahead 30} ( k , {K}_{g} ) ]\end{matrix}$$

In both aforementioned parameter adaptation methods, γ is a parameter adaptation scaling factor used to tune the rate of parameter adaptation. Those skilled in the art will appreciate the tradeoffs associated in choosing a relatively large or small γ value. For instance, forms of adaptive control suitable for use with the embodiments include those described in ASTRÖM AND WITTENMARK, ADAPTIVE CONTROL (Addison-Wesley, 2nd ed. 1995), incorporated herein by reference. In general, a relatively large parameter adaptation scaling factor leads to a faster parameter convergence rate, at the risk of loss of robustness with respect to unmodeled dynamics and other sources of errors. In practice, the choice of γ is determined by examining the effect of different values on the robustness and rate of convergence of the parameters on a many datasets containing retrospective user data. During the retrospective tuning, one may start with the value γ=1, or assign a different starting value based on an error budget analysis of the system.

In other embodiments, the derivation of other controller parameters may follow a similar procedure.

In another embodiment, after the primary controller parameters have been tuned, certain target-related parameters may be tuned to correlate best to specific times of day, times of week, or user-announced events such as health condition, intensity, and duration of physical activity, meal information, and time zone adjustment. Examples of target-related parameters in the context of the controller architecture shown above include the glucose target G_{target} and glucose target rate of change Ġ_{target}.

In another embodiment, instead of using a single set point for a given category (for example, having 100 mg/dL as the single target for glucose, G_{target}), a lower G₁, and upper target Gᵤ range values may be employed, and their values can be made to change over time. Taking the same control action example previously described, the insulin amount delivered to bring current glucose into target, u_{g}(k), is then:

$${u}_{g} ( k ) = \{ \begin{matrix}{K}_{g} \times ( {G}_{CGM} ( k ) - {G}_{u} ) & if {G}_{CGM} ( k ) > {G}_{u} \\ {K}_{g} \times ( {G}_{CGM} ( k ) - {G}_{l} ) & if {G}_{CGM} ( k ) < {G}_{l}\end{matrix} \\ {G}_{u} > {G}_{l}$$

In this case, adaptation is linked to these limits by means of other measurable metrics, such as the number of hypoglycemic and hyperglycemic events associated with using previous limits. In some embodiments, adaptive methods such as the MIT Rule, or other adaptation methods such as the sign-sign algorithm, Least-Squares Error fit or Recursive Least Squares with Exponential Forgetting may be used to continually adapt the target range limits to achieve a good compromise between optimal nominal performance and robustness to uncertainties. In some embodiments, numerical methods such as steepest descent method, simplex method, Newton's method, and the Amoeba method may also be used to improve on the controller parameters.

In addition to considering the optimal parameter for an entire distribution of data, one embodiment uses the above process as applied to subsets of the data to obtain an estimate of a scatter of the particular parameter being estimated. As more and more data is collected, system 10 samples each data point associated with the parameter being identified to obtain a good estimate of the confidence interval of the parameter. This allows for continuous adaptation of the limits of the parameters themselves, providing one method of adapting a higher order aspect. Limiting the allowed range of values of a parameter being adapted is done in order to ensure that any adaptation error would not result in an overly unreasonable estimate of the parameter, and potentially compromise certain safety aspect of the system.

In one embodiment, the limits of an upper glucose target range Gᵤ (that is, target glucose threshold 68 (FIG. 3A)) may be adapted according the above process. For example:

$$\begin{matrix}\frac{d}{d t} {G}_{u} = - γ \frac{\partial }{\partial {G}_{u}} J ( k , {G}_{u} ) \\ = - γ {e}_{ahead 30}^{2} ( k , {G}_{u} ) \frac{\partial }{\partial {G}_{u}} [ {e}_{ahead 30}^{2} ( k , {G}_{u} ) ]\end{matrix}$$

In a further embodiment, the adaptation process may be applied to subsets of the same data, for example, the safety limits on the range of a parameter (for example, G_{uMin} and G_{uMax} of the parameter Gᵤ). For instance, let Gᵤ be allowed to take any value between G_{uMin} and G_{uMax} during its adaptation process, where the upper glucose target range is adjusted to obtain a good tradeoff between optimal and robust performance.

$${G}_{uLimited} = \{ \begin{matrix}{G}_{uMin} & if & {G}_{u} \leq {G}_{uMin} \\ {G}_{uMax} & if & {G}_{u} \geq {G}_{uMax} \\ {G}_{u} & otherwise & \end{matrix}$$

In the beginning, the value G_{uMin} and G_{uMax} are set to certain values (for example, G_{uMin}=100 and G_{uMax=}200 mg/dL) so that during this time, Gᵤ will vary as computed by the adaptation process, but will not exceed these limits. In this example, if it is found from the relevant historical data that whenever Gᵤ takes the value between G_{uMin} (that is, the latest lower limit of the upper limit Gᵤ) and a certain value lower than G_{uMax} (for example, 120 mg/dL) an unacceptable level of hypoglycemic events are found, then the lower limit G_{uMin} (of the upper limit Gᵤ) can be gradually revised to take a new, higher value (for example, 120 mg/dL). For instance, where G_{uMin}=100 and G_{uMax}=200 mg/dL the new limits for the adaptation process of Gᵤ may be set to G_{uMin}=120 mg/dL and G_{uMax}=200 mg/dL respectively. Conversely, if it is found from the relevant historical data that whenever Gᵤ takes on a value between, for instance, 200 mg/dL (the latest upper limit of the upper limit Gᵤ) and 185 mg/dL an unacceptable level of hyperglycemic events is demonstrated then the upper limit G_{uMax} (of the upper limit Gᵤ) can be gradually revised downward to take on a new, lower value of, for instance, 185 mg/dL (setting the new limits for the adaptation process of Gᵤ to G_{uMin}=100 mg/dL and G_{uMax}=185 mg/dL respectively). In some embodiments, the window size of this higher order adaptation process may also be larger than the window size used to adapt Gᵤ itself.

In one embodiment, the manner in which the parameters are improved upon may also be implemented online in a continuous manner, for example, at remote device 50, 52, where the user may or may not be notified of any of the changes. The revisions may also be done at specific time intervals, say, for example, once every 3 months, to account for changes in the subject's circumstances. The revisions may also be done only at specific times as determined by the user and/or the HCP, provided that there is sufficient data to perform the revisions. An example of the latter is whenever the user consults the health care provider for the user's overall diabetes management strategy.

Another embodiment adjusts safety limits (or other types of limits) on variable or parameter values, based on an analysis of the collected data. In one embodiment, a future performance metric generated (for example, y_{ahead30actual}) is used as a reading in the calculation of the trend value in glycemia avoidance analysis. In one embodiment, the metric may signal a high glucose alarm. The control algorithm may also use the metric to limit the total daily insulin delivered to the user. In an embodiment incorporating a separate electronic device 12, data sampled by sensor 31 may be continually monitored and/or processed by processor 14, and stored in memory 16. The data can also be secured on a removable memory medium or other form of secure backup incorporated with device 12, or, for instance, by sending the data to remote device 50, 52 for storage in memory 54. If past data shows that the user has recently (say over the last 3 months) frequently been consuming insulin close to or at a predetermined safety limit, then it may be appropriate to adjust performance limits upward. In one embodiment the system requests the user or HCP to confirm the change in the limit prior to making the change. In other embodiments system 10 automatically changes the limit in accordance with a preprogrammed diabetes management profile or other parameters.

In another aspect of the embodiments, the collected data may be analyzed to improve fault detection parameters in the CGM and/or control algorithm. For instance, the data analysis may determine that for a particular user, sensor dropout is relatively more prevalent, and thus the system will extend the hypo alarm delay parameter beyond what it would be for other users. In another embodiment, the data analysis may indicate a fault in the system such that something needs to be repaired or replaced. For instance, a greater incidence of high frequency variation may be an indication that the CGM transmitter needs to be replaced.

Another aspect of this embodiment, is a means to identify periods of data to be used in the analysis. In this embodiment, the system provides checklists/reminders for tasks that are conducted in order to optimize system performance. For instance, the system notifies the user that it is time to start collecting data periods associated with fasting in order to determine optimal basal rate and some other control parameter determination. In one embodiment, the system 10 may have programmable reminders for users to behave in a prescribed manner for a period of time, for instance, fasting or not exercising, or having a meal, or turning on peak detection. In another embodiment, the system may provide a means for the user to mark these times; for example, an event is logged with a time stamp to mark the start and stop of a data period for analysis and display. The retrospective analysis program at a future time searches this logged data for these time periods and uses them in the appropriate analysis.

The forgoing description of the embodiments of the invention has been presented for the purposes of illustration and description. It is not intended to be exhaustive or to limit the invention to the precise form disclosed. Many modifications and variations are possible in light of the above teaching. It is intended that the scope of the invention not be limited by this detailed description, but by the claims and the equivalents to the claims appended hereto.

Although the present invention has been described in detail with regard to the preferred embodiments and drawings thereof, it should be apparent to those of ordinary skill in the art that various adaptations and modifications of the present invention may be accomplished without departing from the spirit and the scope of the invention. Accordingly, it is to be understood that the detailed description and the accompanying drawings as set forth hereinabove are not intended to limit the breadth of the present invention.

We claim:

1. A system for proactively monitoring glucose levels, the system comprising:
a sensor that measures an indication of glucose and provides levels of glucose over a monitoring time period;
a memory having stored therein a safe range of glucose;
a device that provides a meal signal indicating that a meal has been consumed, the device also providing a medication administration signal indicating an amount of medication and a time that it was administered;
a processor configured to receive the meal signal, levels of glucose, and the safe range, wherein upon receiving the meal signal, the processor is further configured to:
monitor the levels of glucose beginning after the meal signal;
compare the levels of glucose to the safe range;
if a monitored glucose level is outside the safe range, determine a post-prandial vertex of the glucose level, and
once the vertex is determined, provide an action to return the glucose level to a target glucose level within the safe range, wherein the action includes consideration of a user parameter.

2. The system for proactively monitoring glucose levels of claim 1, wherein the vertex is a peak and the action provided by the processor includes calculating a dose of medication sufficient to reduce the glucose level more rapidly to the target glucose level within the safe range and to decrease an amount of high glucose exposure than if the action was not taken.

3. The system for proactively monitoring glucose levels of claim 1, wherein the vertex is a valley and the action provided by the processor includes calculating an amount of carbohydrates sufficient to increase the glucose level more rapidly to the target glucose level within the safe range than if the action was not taken.

4. The system for proactively monitoring glucose levels of claim 1, wherein the vertex is a valley and the action by the processor includes calculating a reduction in a basal rate of a medication administration sufficient to raise the glucose level more rapidly to the target glucose level within the safe range than if the action was not taken.

5. The system for proactively monitoring glucose levels of claim 1, wherein the processor is configured to delay monitoring the levels of glucose for a selected time period after receiving the meal signal.

6. The system for proactively monitoring glucose levels of claim 1, wherein the the processor is configured to determine the vertex by comparing a first and a second glucose level trend, the vertex being at the point at which the first and second glucose level trends diverge.

7. The system for proactively monitoring glucose levels of claim 1, wherein the processor is configured to determine the vertex by comparing a first and a second glucose rate of change, the vertex being at the point at which the signs of the first and second glucose rate of change diverge.

8. The system for proactively monitoring glucose levels of claim 1, wherein the processor is further configured to:
identify a first and second set of sensed glucose readings over at least a portion of the monitoring time period to determine a first and second glucose level trend, the vertex being determined by comparing a first and a second glucose level trend, and to
calculate a first and second set of smoothed glucose values representative of the first and second set of sensed glucose readings prior to determining the first glucose level trend, and to determine the first and second glucose level trend as a function of first and second set of smoothed glucose values.

9. The system for proactively monitoring glucose levels of claim 8, wherein the first and second glucose level trend is a trend in a first and a second glucose rate of change.

10. The system for proactively monitoring glucose levels of claim 8, wherein the user parameter is selected from a group consisting of an insulin action time, a level of insulin-on-board, an insulin sensitivity factor

11. A method for proactively monitoring glucose levels, the method comprising:
measuring an indication of glucose and providing levels of glucose over a monitoring time period;
storing in a memory a safe range of glucose;
providing a meal signal indicating that a meal has been consumed;
providing a medication administration signal indicating an amount of medication and a time that it was administered;
receiving the meal signal, levels of glucose, and the safe range, wherein upon receiving the meal signal:
monitoring the levels of glucose beginning after the meal signal;
comparing the levels of glucose to the safe range;
if a monitored glucose level is outside the safe range, determining a post-prandial vertex of the glucose level, and
once the vertex is determined, providing an action to return the glucose level to a target glucose level within the safe range, wherein the action includes consideration of a user parameter.

12. The method for proactively monitoring glucose levels of claim 11, wherein the vertex is a peak and providing the action includes calculating a dose of medication sufficient to reduce the glucose level more rapidly to the target glucose level within the safe range and to decrease an amount of high glucose exposure than if the action was not taken.

13. The method for proactively monitoring glucose levels of claim 11, wherein the vertex is a valley and providing the action includes calculating an amount of carbohydrates sufficient to increase the glucose level more rapidly to the target glucose level within the safe range than if the action was not taken.

14. The method for proactively monitoring glucose levels of claim 11, wherein the vertex is a valley and providing the action includes calculating a reduction in a basal rate of a medication administration sufficient to raise the glucose level more rapidly to the target glucose level within the safe range than if the action was not taken.

15. The method for proactively monitoring glucose levels of claim 11, further comprising:
delaying monitoring the levels of glucose for a selected time period after receiving the meal signal.

16. The method for proactively monitoring glucose levels of claim 11, further comprising:
determining the vertex by comparing a first and a second glucose level trend, the vertex being at the point at which the first and second glucose level trends diverge.

17. The method for proactively monitoring glucose levels of claim 11, further comprising:
determining the vertex by comparing a first and a second glucose rate of change, the vertex being at the point at which the signs of the first and second glucose rate of change diverge.

18. The method for proactively monitoring glucose levels of claim 11, further comprising:
identifying a first and second set of sensed glucose readings over at least a portion of the monitoring time period to determine a first and second glucose level trend, the vertex being determined by comparing a first and a second glucose level trend; and
calculating a first and second set of smoothed glucose values representative of the first and second set of sensed glucose readings prior to determining the first glucose level trend, and to determine the first and second glucose level trend as a function of first and second set of smoothed glucose values.

19. The method for proactively monitoring glucose levels of claim 18, wherein the first and second glucose level trend is a trend in a first and a second glucose rate of change.

20. The method for proactively monitoring glucose levels of claim 18, wherein the user parameter is selected from a group consisting of an insulin action time, a level of insulin-on-board, and an insulin sensitivity factor.

21. A method for integrated diabetes management, comprising:
receiving at a controller a meal indication input;
receiving at the controller and after the meal indication input a first set of sensed glucose readings in a user from a continuous glucose sensor;
receiving at the controller a second set of sensed glucose readings in the user from the continuous glucose sensor, wherein the second set of sensed glucose readings begin later in time than the first set of sensed glucose readings and at least one of the second set of sensed readings is above a high glucose threshold;
recording the first and second set of glucose readings on a memory medium;
determining a first glucose level trend in the user as a function of the first set of glucose signals;
determining a second glucose level trend in the user as a function of the second set of glucose signals;
sending a signal indicative of a user glucose level peak from the controller to a display when the first glucose level trend and second glucose level trend diverge; and
wherein the signal includes an amount of insulin required to reduce an insulin action time necessary to reduce a third set of sensed glucose readings below the high glucose threshold and to minimize a high glucose exposure.

22. A method of claim 21, wherein the first glucose level trend is a first rate of change in the first set of sensed glucose readings in the user, and
wherein the second glucose level trend is a second rate of change in the second set of sensed glucose readings in the user.

23. A method of claim 22, further comprising:
calculating a first set of smoothed glucose values representative of the first set of sensed glucose readings prior to determining the first glucose level trend, wherein the first glucose level trend is determined as a function of first set of smoothed glucose values; and
calculating a second set of smoothed glucose value representative of the second set of sensed glucose readings prior to determining the second glucose level trend; wherein the second glucose level trend is determined as a function of second set of smoothed glucose values.

24. A method of claim 23, wherein the first glucose level trend is a value in the second set of smoothed glucose values.

25. A method for adjusting a control parameter used in an integrated diabetes management (IDM) system, the method comprising:
storing in a memory a control parameter;
providing a medication administration signal representative of an amount of medication as a function of the control parameter;
measuring levels of glucose over a monitoring time period;
determining a performance metric as a function of the levels of glucose and the medication administration signal over a first window of time; and,
if the performance metric is outside an expected range, adjusting the control parameter to adjust the amount of medication and to bring the performance metric inside the expected range.

26. The method for adjusting a control parameter of claim 25, wherein the expected range is determined as a function of an empirical series of performance metrics over a selected window of time.

27. The method for adjusting a control parameter of claim 26, wherein the selected window starts before a beginning of the first window.

28. The method for adjusting a control parameter of claim 26, wherein the first window at least partially overlaps the selected window in time.

29. The method for adjusting a control parameter of claim 26, wherein the first and selected window do not overlap in time.

30. The method for adjusting a control parameter of claim 25, wherein the performance metric includes an average glucose expected from a present time to a future time.

31. The method for adjusting a control parameter of claim 25, wherein the performance metric includes a slope of the levels of glucose over a selected window of time.

32. The method for adjusting a control parameter of claim 25, further comprising:
providing a control limit on the control parameter;
monitoring a set of control parameters;
determining one or more physiological events over a selected window of time;
if the one or more physiological events is outside an unacceptable level of physiological events, adjusting the control limit to avoid a further unacceptable physiological event.

33. The method for adjusting a control parameter of claim 32, wherein the control limit is determined as a function of a scatter of the set of control parameters over a selected time period.

34. The method for adjusting a control parameter of claim 32, wherein the unacceptable physiological events includes a number of hypoglycemic events in a user.

35. The method for adjusting a control parameter of claim 32, wherein the unacceptable physiological events includes a number of hyperglycemic events in a user.

36. The method for adjusting a control parameter of claim 25, further comprising:
if, over a selected window of time, consecutively computed insulin commands are negative for more than a predetermined duration, providing an alarm including an indication of a high likelihood of a hypoglycemic event.

37. The method for adjusting a control parameter of claim 36, further comprising:
providing a request to enter a manual glucose reading using a strip port, wherein the control parameter is adjusted as a function of the manual glucose reading.

38. The method for adjusting a control parameter of claim 37, further comprising:
if the manual glucose reading confirms the consecutively computed insulin command, providing a second alarm including a suggestion to ingest an amount of carbohydrates.

39. The method for adjusting a control parameter of claim 25, further comprising:
if, over a selected window of time, a first contiguous area formed by consecutively computed insulin commands exceeds a second area formed by an integral of a predetermined duration, providing an alarm including an indication of a high likelihood of a hypoglycemic event.

40. The method for adjusting a control parameter of claim 39, further comprising:
providing a request to enter a manual glucose reading using a strip port, wherein the control parameter is adjusted as a function of the manual glucose reading.

41. The method for adjusting a control parameter of claim 40, further comprising:
if the manual glucose reading confirms the consecutively computed insulin command, providing a second alarm including a suggestion to ingest an amount of carbohydrates.

42. A system for adjusting a control parameter used in an integrated diabetes management (IDM) system, the system comprising:
a memory having a control parameter stored thereon;
a device configured to provide a medication administration signal representative of an amount of medication as a function of the control parameter, the device being further configured to measure levels of glucose over a monitoring time period;
a processor configured to determine a performance metric as a function of the levels of glucose and the medication administration signal over a first window of time, such that,
if the performance metric is outside an expected range, the processor is further configured to adjust the control parameter to adjust the medication administration signal and to bring the performance metric inside the expected range.

43. The system for adjusting a control parameter of claim 42, the processor being further configured to determine the expected range as a function of an empirical series of performance metrics over second window of time.

44. The system for adjusting a control parameter of claim 43, wherein the selected window starts before a beginning of the first window.

45. The system for adjusting a control parameter of claim 43, wherein the first window at least partially overlaps the selected window in time.

46. The system for adjusting a control parameter of claim 43, wherein the first and selected window do not overlap in time.

47. The system for adjusting a control parameter of claim 42, wherein the performance metric includes an average glucose expected from a present time to a future time.

48. The system for adjusting a control parameter of claim 42, wherein the performance metric includes a slope of the levels of glucose over a selected window of time.

49. The system for adjusting a control parameter of claim 42, further comprising:
providing a control limit on the control parameter;
monitoring a set of control parameters;
determining one or more physiological events over a selected window of time;
if the one or more physiological events is outside an unacceptable level of physiological events, adjusting the control limit to avoid a further unacceptable physiological event.

50. The system for adjusting a control parameter of claim 49, wherein the control limit is determined as a function of a scatter of the set of control parameters over a selected time period.

51. The system for adjusting a control parameter of claim 49, wherein the unacceptable physiological events includes a number of hypoglycemic events in a user.

52. The system for adjusting a control parameter of claim 49, wherein the unacceptable physiological events includes a number of hyperglycemic events in a user.

53. The system for adjusting a control parameter of claim 42, further comprising:
if, over a selected window of time, consecutively computed insulin commands are negative for more than a predetermined duration, providing an alarm including an indication of a high likelihood of a hypoglycemic event.

54. The system for adjusting a control parameter of claim 53, further comprising:
providing a request to enter a manual glucose reading using a strip port, wherein the control parameter is adjusted as a function of the manual glucose reading.

55. The system for adjusting a control parameter of claim 54, further comprising:
if the manual glucose reading confirms the consecutively computed insulin command, providing a second alarm including a suggestion to ingest an amount of carbohydrates.

56. The system for adjusting a control parameter of claim 42, further comprising:
if, over a selected window of time, a first contiguous area formed by consecutively computed insulin commands exceeds a second area formed by an integral of a predetermined duration, providing an alarm including an indication of a high likelihood of a hypoglycemic event.

57. The system for adjusting a control parameter of claim 56, further comprising:
providing a request to enter a manual glucose reading using a strip port, wherein the control parameter is adjusted as a function of the manual glucose reading.

58. The system for adjusting a control parameter of claim 57, further comprising:
if the manual glucose reading confirms the consecutively computed insulin command, providing a second alarm including a suggestion to ingest an amount of carbohydrates.

59. A method for integrated diabetes management, comprising:
providing a controller with a control parameter;
sampling at the controller a first set of insulin delivery commands over a first window of time;
sampling at the controller a second set of insulin delivery commands over a second window of time, wherein the first and second insulin delivery commands provide information to deliver an amount of insulin and are determined as a factor of a difference between a sensed glucose value and a target glucose value and a control parameter;
determining a first performance metric as a function of the first set of insulin delivery commands;
determining a second performance metric as a function of the second set of insulin delivery commands;
adjusting the control parameter as a function of the first and second performance metric to generate an adjusted control parameter;
determining a future insulin delivery command as a function of the adjusted control parameter; and
graphically displaying information representative of the future insulin delivery command on a graphic display.

60. The method of claim 59, wherein the first and second insulin delivery commands are further determined as a factor of a difference between the latest CGM rate of change and a target rate of change.

61. The method of claim 60, further comprising:
delivering a first insulin amount to a user; and
delivering a second insulin amount to the user based on the second delivery command, wherein the second insulin amount is based on the difference between a present value of glucose and a target value of glucose.

62. The method of claim 61, wherein the present value is a present CGM value and the target value is a target CGM value.

63. The method of claim 61, wherein the present value is a present CGM rate of change, and the target value is a target CGM rate of change.

64. The method of claim 61, wherein the present value is a present insulin-on-board, and the target value is a target insulin-on-board.

65. The method of claim 61, further comprising:
tuning the target value to correlate to a time period or physical condition.

66. The method of claim 59, further comprising:
determining an insulin delivery command as a function of the performance metric and the adjusted control parameter;
prompting a user at a terminal to confirm the insulin delivery amount; and
delivering the insulin delivery amount to a user upon receiving a confirmation from the user at the terminal.
